SEQUENCE LISTING (12 sequences)

SEQ ID NO: 1 - DNA, 2817 bp, Corynebacterium glutamicum
  ATCC14752
aatgaaataatcccctcaccaactggcgacattcaaacaccgtttcatttccaaacatcgagccaagggaaaagaaagcccctaagccccgtgttattaaatggagactctttggagacctcaagccaaaaaggggcattttcattaagaaaatacccctttgacctggtgttattgagctggagaagagacttgaactctcaacctacgcattacaagtgcgttgcgctgccaattgcgccactccagcaccgcagatgctgatgatcaacaactacgaatacgtatcttagcgtatgtgtacatcacaatggaattcggggctagagtatctggtgaaccgtgcataaacgacctgtgattggactctttttccttgcaaaatgttttccagcggatgttgagttttgcgacccttcgtggccgcatttcaacagttgacgctgcaaaagccgcacctccgccatcgccactagccccgattgatctcactgaccatagtcaagtggccggtgtgatgaatttggctgcgagaattggcgatattttgctttcttcaggtacgtcaaacagtgataccaaggtgcaagttcgagcggtgacctctgcgtatggcctgtactatacgcatgtggatatcacgttgaatacgatcaccatcttcaccaacatcggtgtggagaggaagatgccggtcaacgtgtttcatgttgtgggcaagttggacaccaacttctccaaactgtctgaggttgaccgtttgatccgttccattcaggctggtgctaccccgcctgaggttgccgagaaaattctggacgagttggagcaatcgcctgcgtcttatggtttccctgttgcgttgcttggctgggcaatgatgggtggcgctgttgctgtgctgttgggtggtggatggcaggtttccctaattgcttttattaccgcgttcacgatcattgccacgacgtcatttttgggaaagaagggtttgcctactttcttccaaaatgttgttggtggttttattgccacgctgcctgcatcgattgcttattctttggcgttgcaatttggtcttgagatcaaaccgagccagatcatcgcatctggaattgttgtgctgttggcaggtttgacacttgtgcaatctctgcaggacggcatcacgggcgctccggtgacagcaagtgcacgattttttgaaacactcctgtttaccggcggcattgttgctggcgtgggtttgggcattcagctttctgaaatcttgcatgtcatgttgcctgccatggagtccgctgcagcacctaattattcgtctacattcgcccgcattatcgctggtggcgtcaccgcagcggccttcgcagtgggttgttacgcggagtggtcctcggtgattattgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccttgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcaaagattcggtaataaaaggtaaaaatcaacctgcttaggcgtctttcgcttaaatagcgtagaatatcgggtcgatcgcttttaaacactcaggaggatccttgccggccaaaatcacggacactcgtcccaccccagaatcccttcacgctgttgaagaggaaaccgcagccggtgcccgcaggattgttgccacctattctaaggacttcttcgacggcgtcactttgatgtgcatgctcggcgttgaacctcagggcctgcgttacaccaaggtcgcttctgaacacgaggaagctcagccaaagaaggctacaaagcggactcgtaaggcaccagctaagaaggctgctgctaagaaaacgaccaagaagaccactaagaaaactactaaaaagaccaccgcaaagaagaccacaaagaagtcttaagccggatcttatatggatgattccaatagctttgtagttgttgctaaccgtctgccagtggatatgactgtccacccagatggtagctatagcatctcccccagccccggtggccttgtcacggggctttcccccgttctggaacaacatcgtggatgttgggtcggatggcctggaactgtagatgttgcacccgaaccatttcgaacagatacgggtgttttgctgcaccctgttgtcctcactgcaagtgactatgaaggcttctacgagggcttttcaaacgcaacgctgtggcctcttttccacgatttgattgttactccggtgtacaacaccgattggtggcatgcgtttcgggaagtaaacctcaagttcgctgaagccgtgagccaagtggcggcacacggtgccactgtgtgggtgcaggactatcagctgttgctggttcctggcattttgcgccagatgcgccctgatttgaagatcggtttcttcctccacattcccttcccttcccctgatctgttccgtcagctgc

SEQ ID NO: 2 - protein, 489 aa, Corynebacterium glutamicum
  ATCC14752
MLSFATLRGRISTVDAAKAAPPPSPLAPIDLTDHSQVAGVMNLAARIGDILLSSGTSNSDTKVQVRAVTSAYGLYYTHVDITLNTITIFTNIGVERKMPVNVFHVVGKLDTNFSKLSEVDRLIRSIQAGATPPEVAEKILDELEQSPASYGFPVALLGWAMMGGAVAVLLGGGWQVSLIAFITAFTIIATTSFLGKKGLPTFFQNVVGGFIATLPASIAYSLALQFGLEIKPSQIIASGIVVLLAGLTLVQSLQDGITGAPVTASARFFETLLFTGGIVAGVGLGIQLSEILHVMLPAMESAAAPNYSSTFARIIAGGVTAAAFAVGCYAEWSSVIIAGLTALMGSAFYYLFVVYLGPVSAAAIAATAVGFTGGLLARRFLIPPLIVAIAGITPMLPGLAIYRGMYATLNDQTLMGFTNIAVALATASSLAAGVVLGEWIARRLRRPPRFNPYRAFTKANEFSFQEEAEQNQRRQRKRPKTNQRFGNKR

SEQ ID NO: 3 - DNA, 1909 bp, Corynebacterium glutamicum
  ATCC13032
agcttgcatgcctgcaggtcgactctagaggatcccccccctttgacctggtgttattgagctggagaagagacttgaactctcaacctacgcattacaagtgcgttgcgctgccaattgcgccactccagcaccgcagatgctgatgatcaacaactacgaatacgtatcttagcgtatgtgtacatcacaatggaattcggggctagagtatctggtgaaccgtgcataaacgacctgtgattggactctttttccttgcaaaatgttttccagcggatgttgagttttgcgacccttcgtggccgcatttcaacagttgacgctgcaaaagccgcacctccgccatcgccactagccccgattgatctcactgaccatagtcaagtggccggtgtgatgaatttggctgcgagaattggcgatattttgctttcttcaggtacgtcaaatagtgacaccaaggtacaagttcgagcagtgacctctgcgtacggtttgtactacacgcacgtggatatcacgttgaatacgatcaccatcttcaccaacatcggtgtggagaggaagatgccggtcaacgtgtttcatgttgtaggcaagttggacaccaacttctccaaactgtctgaggttgaccgtttgatccgttccattcaggctggtgcgaccccgcctgaggttgccgagaaaatcctggacgagttggagcaatcccctgcgtcttatggtttccctgttgcgttgcttggctgggcaatgatgggtggtgctgttgctgtgctgttgggtggtggatggcaggtttccctaattgcttttattaccgcgttcacgatcattgccacgacgtcatttttgggaaagaagggtttgcctactttcttccaaaatgttgttggtggttttattgccacgctgcctgcatcgattgcttattctttggcgttgcaatttggtcttgagatcaaaccgagccagatcatcgcatctggaattgttgtgctgttggcaggtttgacactcgtgcaatctctgcaggacggcatcacgggcgctccggtgacagcaagtgcacgatttttcgaaacactcctgtttaccggcggcattgttgctggcgtgggtttgggcattcagctttctgaaatcttgcatgtcatgttgcctgccatggagtccgctgcagcacctaattattcgtctacattcgcccgcattatcgctggtggcgtcaccgcagcggccttcgcagtgggttgttacgcggagtggtcctcggtgattattgcggggcttactgcgctgatgggttctgcgttttattacctcttcgttgtttatttaggccccgtctctgccgctgcgattgctgcaacagcagttggtttcactggtggtttgcttgcccgtcgattcttgattccaccgttgattgtggcgattgccggcatcacaccaatgcttccaggtctagcaatttaccgcggaatgtacgccaccctgaatgatcaaacactcatgggtttcaccaacattgcggttgctttagccactgcttcatcacttgccgctggcgtggttttgggtgagtggattgcccgcaggctacgtcgtccaccacgcttcaacccataccgtgcatttaccaaggcgaatgagttctccttccaggaggaagctgagcagaatcagcgccggcagagaaaacgtccaaagactaatcagagattcggtaataaaaggtaaaaatcaacctgcttaggcgtctttcgcttaaatagcgtagaatatcgggtcgatcgcttttaaacactcaggaggatccttgccggccaaaatcacggacactcgtcccaccccagaatcccttcacgctgttgaagaggaaaccgcagccggggtaccg

SEQ ID NO: 4 - protein, 489 aa, Corynebacterium glutamicum; identical to SEQ ID NO: 2
  ATCC13032

SEQ ID NO: 5 - DNA, 23 bp, Artificial Sequence
  Description of Artificial Sequence Artificial primer
cgggtctacaccgctagcccagg

SEQ ID NO: 6 - DNA, 20 bp, Artificial Sequence
  Description of Artificial Sequence Artificial primer
cggtgccttatccattcagg

SEQ ID NO: 7 - DNA, 22 bp, Artificial Sequence
  Description of Artificial Sequence Artificial primer
cccctttgacctggtgttattg

SEQ ID NO: 8 - DNA, 18 bp, Artificial Sequence
  Description of Artificial Sequence Artificial primer
cggctgcggtttcctctt

SEQ ID NO: 9 - DNA, 17 bp, Artificial Sequence
  Description of Artificial Sequence Artificial primer
gtaaaacgacggccagt

SEQ ID NO: 10 - DNA, 19 bp, Artificial Sequence
  Description of Artificial Sequence Artificial primer
ggaaacagctatgaccatg

SEQ ID NO: 11 - DNA, 20 bp, Artificial Sequence
  Description of Artificial Sequence Artificial primer
tcgcgacctataagtttggg

SEQ ID NO: 12 - DNA, 20 bp, Artificial Sequence
  Description of Artificial Sequence Artificial primer
aataccagcccttgttcgtg

(19) United States
(12) Patent Application Publication
(10) Pub. No.: US 2002/0155551 A1
(43) Pub. Date: Oct. 24, 2002

(54) Process for the fermentative preparation of L-threonine

(75) Inventor: Mechthild Rieping, Bielefeld (DE)

Correspondence Address: PILLSBURY WINTHROP LLP, 1600 TYSONS BOULEVARD, MCLEAN, VA, 22102, US

(73) Assignee: DEGUSSA AG

(21) Appl. No.: 09/834,721

(22) Filed: Apr. 16, 2001

Related U.S. Application Data
(60) Provisional application No. 60/229,328, filed on Sep. 1, 2000.

(30) Foreign Application Priority Data
May 27, 2000 (DE) 100 26 494.8
Jan. 23, 2001 (DE) 101 02 823.7

(51) Int. Cl. C12P 13/04; C12N 1/21

(52) U.S. Cl. 435/106; 435/252.3

(57) ABSTRACT

Process for the fermentative preparation of L-threonine The invention provides a process for the fermentative preparation of L-threonine using Enterobacteriaceae which in particular already produce L-threonine and in which the nucleotide sequence(s) of coryneform bacteria which code(s) for the thrE gene are enhanced, in particular over-expressed.

This invention relates to a process for the fermentative preparation of L-threonine using Enterobacteriaceae in which the thrE gene of coryneform bacteria is enhanced.

### PRIOR ART

L-Threonine is used in animal nutrition, in human medicine and in the pharmaceuticals industry. It is known that L-threonine can be prepared by fermentation of strains of Enterobacteriaceae, in particular Escherichia coli and Serratia marcescens. Because of their great importance, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form, by e.g. ion exchange chromatography, or the intrinsic output properties of the microorganism itself.

Methods of mutagenesis, selection and mutant selection are used to improve the output properties of these microorganisms. Strains which are resistant to antimetabolites, such as e.g. the threonine analogue α-amino-β-hydroxyvaleric acid (AHV), or are auxotrophic for amino acids of regulatory importance and produce L-threonine are obtained in this manner.

Methods of the recombinant DNA technique have also been employed for some years for improving the strain of Enterobacteriaceae strains which produce L-threonine, by amplifying individual threonine biosynthesis genes and investigating the effect on the L-threonine production.

### OBJECT OF THE INVENTION

The inventors had the object of providing new measures for improved fermentative preparation of L-threonine.

### DESCRIPTION OF THE INVENTION

The invention provides a process for the fermentative preparation of L-threonine using Enterobacteriaceae which in particular already produce L-threonine and in which the nucleotide sequence(s) of coryneform bacteria which code(s) for the thrE gene are enhanced, in particular over-expressed.

In particular, the process is a process for the preparation of L-threonine, which comprises carrying out the following steps:

a) fermentation of microorganisms of the family Enterobacteriaceae in which at least the thrE gene of coryneform bacteria is enhanced (over-expressed), optionally in combination with further genes,

b) concentration of the L-threonine in the medium or in the cells of the microorganisms of the family Enterobacteriaceae, and

c) isolation of the L-threonine.

The term “enhancement” in this connection describes the increase in the intracellular activity of one or more enzymes or proteins in a microorganism which are coded by the corresponding DNA, for example by increasing the number of copies of the gene or genes, using a potent promoter or a gene which codes for a corresponding enzyme or protein with a high activity, and optionally combining these measures.

The microorganisms which the present invention provides can prepare L-threonine from glucose, sucrose, lactose, fructose, maltose, molasses, starch, cellulose or from glycerol and ethanol. They are representatives of Enterobacteriaceae, in particular of the genera Escherichia and Serratia. Of the genus Escherichia the species Escherichia coli and of the genus Serratia the species Serratia marcescens are to be mentioned in particular.

Suitable L-threonine-producing strains of the genus Escherichia, in particular of the species Escherichia coli, are, for example

Escherichia coli TF427

Escherichia coli H4578

Escherichia coli KY10935

Escherichia coli VNIIgenetika MG-442

Escherichia coli VNIIgenetika M1

Escherichia coli VNIIgenetika 472T23

Escherichia coli BKIIM B-3996

Escherichia coli kat 13

Escherichia coli KCCM-10132

Suitable L-threonine-producing strains of the genus Serratia, in particular of the species Serratia marcescens, are, for example

Serratia marcescens HNr21

Serratia marcescens TLr156

Serratia marcescens T2000

It has been found that Enterobacteriaceae produce L-threonine in an improved manner after over-expression of the thrE gene of coryneform bacteria which codes for threonine export.

Nucleotide sequences of the thrE gene of coryneform bacteria are shown in SEQ ID No 1 and SEQ ID No 3 and the resulting amino acid sequences of the export proteins are shown in SEQ ID No 2 and SEQ ID No 4.

The thrE gene shown in SEQ ID No 1 and SEQ ID No 3 can be used according to the invention. Alleles of the thrE gene of coryneform bacteria which result from the degeneracy of the genetic code or due to sense mutations of neutral function can furthermore be used.

To achieve an over-expression, the number of copies of the corresponding genes can be increased, or the promoter and regulation region or the ribosome binding site upstream of the structural gene can be mutated. Expression cassettes which are incorporated upstream of the structural gene act in the same way. By inducible promoters, it is additionally possible to increase the expression in the course of fermentative L-threonine production. The expression is likewise improved by measures to prolong the life of the m-RNA. Furthermore, the enzyme activity is also increased by preventing the degradation of the enzyme protein. The genes or gene constructions can either be present in plasmids with a varying number of copies, or can be integrated and amplified in the chromosome. Alternatively, an over-expression of the genes in question can furthermore be achieved by changing the composition of the media and the culture procedure.

Instructions in this context can be found by the expert, inter alia, in Chang and Cohen (Journal of Bacteriology 134:1141-1156 (1978)), in Hartley and Gregori (Gene 13:347-353 (1981)), in Amann and Brosius (Gene 40:183-190 (1985)), in de Broer et al. (Proceedings of the National of Sciences of the United States of America 80:21-25 (1983)), in LaVallie et al. (BIO/TECHNOLOGY 11, 187-193 (1993)), in PCT/US97/13359, in Llosa et al. (Plasmid 26:222-224 (1991)), in Quandt and Klipp (Gene 80:161-169 (1989)), in Hamilton (Journal of Bacteriology 171:4617-4622 (1989), in Jensen and Hammer (Biotechnology and Bioengineering 58, 191-195 (1998) and in known textbooks of genetics and molecular biology.

Plasmid vectors which are capable of replication in Enterobacteriaceae, such as e.g. cloning vectors derived from pACYC184 (Bartolomé et al.; Gene 102, 75-78 (1991)), pTrc99A (Amann et al.; (Gene 69:301-315 (1988)), or pSC101 derivatives (Vocke and Bastia, Proceedings of the National Academy of Science USA 80 (21):6557-6561 (1983)) can be used. A strain transformed with a plasmid vector where the plasmid vector carries the nucleotide sequence which codes for the thrE gene of coryneform bacteria can be employed in a process according to the invention.

In addition, it may be advantageous for the production of L-threonine with strains of the family Enterobacteriaceae to over-express one or more enzymes of the known threonine biosynthesis pathway or enzymes of anaplerotic metabolism or enzymes for the production of reduced nicotinamide adenine dinucleotide phosphate, in addition to the thrE gene of coryneform bacteria. Thus, for example

at the same time the thrABC operon which codes for aspartate kinase, homoserine dehydrogenase, homoserine kinase and threonine synthase (U.S. Pat. No. 4,278,765), or

at the same time the pyc gene which codes for pyruvate carboxylase (DE-A-19 831 609), or

at the same time the pps gene which codes for phosphoenol pyruvate synthase (Molecular and General Genetics 231:332 (1992)), or

at the same time the ppc gene which codes for phosphoenol pyruvate carboxylase (Gene 31:279-283 (1984)), or

at the same time the genes pntA and pntB which code for transhydrogenase (European Journal of Biochemistry 158:647-653 (1986)), or

at the same time the gdhA gene which codes for glutamate dehydrogenase (GGene 27:193-199 (1984)), or

at the same time the rhtB gene which imparts L-homoserine resistance (EP-A-0994190)

can be enhanced, in particular over-expressed.

In addition to over-expression of the thrE gene it may furthermore be advantageous, for the production of L-threonine, to eliminate undesirable side reactions, such as e.g. threonine dehydrogenase (Nakayama: “Breeding of Amino Acid Producing Microorganisms”, in: Overproduction of Microbial Products, Krumphanzl, Sikyta, Vanek (eds.), Academic Press, London, UK, 1982 and Bell and Turner, Biochemical Journal 156, 449-458 (1976)). Bacteria in which the metabolic pathways which reduce the formation of L-threonine are at least partly eliminated can be employed in the process according to the invention.

The microorganisms produced according to the invention can be cultured in the batch process (batch culture) or in the fed batch process (feed process). A summary of known culture methods is described in the textbook by Chmiel (Bioprozesstechnik 1. Einführung in die Bioverfahrenstechnik [Bioprocess Technology 1. Introduction to Bioprocess Technology (Gustav Fischer Verlag, Stuttgart, 1991)) or in the textbook by Storhas (Bioreaktoren und periphere Einrichtungen [Bioreactors and Peripheral Equipment] (Vieweg Verlag, Braunschweig/Wiesbaden, 1994)).

The culture medium to be used must meet the requirements of the particular strains in a suitable manner. Descriptions of culture media for various microorganisms are contained in the handbook “Manual of Methods for General Bacteriology” of the American Society for Bacteriology (Washington D.C., USA, 1981). Sugars and carbohydrates, such as e.g. glucose, sucrose, lactose, fructose, maltose, molasses, starch and cellulose, oils and fats, such as e.g. soya oil, sunflower oil, groundnut oil and coconut fat, fatty acids, such as e.g. palmitic acid, stearic acid and linoleic acid, alcohols, such as e.g. glycerol and ethanol, and organic acids, such as e.g. acetic acid, can be used as the source of carbon. These substance can be used individually or as a mixture. Organic nitrogen-containing compounds, such as peptones, yeast extract, meat extract, malt extract, corn steep liquor, soya bean flour and urea, or inorganic compounds, such as ammonium sulphate, ammonium chloride, ammonium phosphate, ammonium carbonate and ammonium nitrate, can be used as the source of nitrogen. The sources of nitrogen can be used individually or as a mixture. Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate or iron sulfate, which are necessary for growth. Finally, essential growth substances, such as amino acids and vitamins, can be employed in addition to the abovementioned substances. Suitable precursors can moreover be added to the culture medium. The starting substances mentioned can be added to the culture in the form of a single batch, or can be fed in during the culture in a suitable manner.

Basic compounds, such as sodium hydroxide, potassium hydroxide, ammonia or aqueous ammonia, or acid compounds, such as phosphoric acid or sulfuric acid, can be employed in a suitable manner to control the pH. Antifoams, such as e.g. fatty acid polyglycol esters, can be employed to control the development of foam. Suitable substances having a selective action, e.g. antibiotics, can be added to the medium to maintain the stability of plasmids. To maintain aerobic conditions, oxygen or oxygen-containing gas mixtures, such as e.g. air, are introduced into the culture. The temperature of the culture is usually 25° C. to 45° C., and preferably 30° C. to 40° C. Culturing is continued until a maximum of L-threonine has formed. This target is usually reached within 10 hours to 160 hours.

The analysis of L-threonine can be carried out by anion exchange chromatography with subsequent ninhydrin derivatization, as described by Spackman et al. (Analytical Chemistry, 30, (1958), 1190), or it can take place by reversed phase HPLC as described by Lindroth et al. (Analytical Chemistry (1979) 51:. 1167-1174).

The following microorganism has been deposited at the Deutsche Sammlung für Mikrorganismen und Zellkylturen (DSMZ=German Collection of Microorganisms and Cell Cultures, Braunschweig, Germany) in accordance with the Budapest Treaty:

Brevibacterium flavum strain DM368-2 pZ1thrE as DSM 12840

The plasmid pZ1thrE contains the thrE gene of Corynebacterium glutamicum ATCC13032.

The present invention is explained in more detail in the following with the aid of embodiment examples.

The isolation of plasmid DNA from Escherichia coli and all techniques of restriction, Klenow and alkaline phosphatase treatment were carried out by the method of Sambrook et al. (Molecular cloning—A laboratory manual (1989) Cold Spring Harbour Laboratory Press). The transformation of Escherichia coli was carried out by the method of Chung et al. (Proceedings of the National Academy of Sciences of the United States of America USA (1989) 86: 2172-2175).

The incubation temperature during the preparation of E. coli strains and transformants was 37° C. In the gene replacement process of Hamilton et al. (Journal of Bacteriology (1989) 171: 4617-4622) temperatures of 30° C. and 44° C. were used.

### EXAMPLE 1

Cloning and Sequencing of the thrE Gene of Corynebacterium glutamicum ATCC14752

1. Transposon Mutagenesis and Mutant Selection

The strain Corynebacterium glutamicum ATCC14752ΔilvA was subjected to mutagenesis with the transposon Tn5531, the sequence of which is deposited under accession number U53587 in the nucleotide databank of the National Center for Biotechnology Information (Bethesda, USA). The incorporation of a deletion in the ilvA gene of Corynebacterium glutamicum ATCC14752 was carried out with the gene exchange system described by Schäfer et al. (Gene (1994) 145: 69-73). For this, an inactivation vector pK19mobsacBΔilvA constructed by Sahm et al. (Applied and Environmental Microbiology (1999) 65: 1973-1979) was used for the deletion. The methylase-defective Escherichia coli strain SCS110 (Jerpseth and Kretz, STRATEGIES in molecular biology 6, 22, (1993)) from Stratagene (Heidelberg, Germany) was first transformed with 200 ng of the vector pK19mobsacBΔilvA. Transformants were identified with the aid of their kanamycin resistance on LB agar plates containing 50 μg/mL kanamycin. The plasmid pK19mobsacBΔilvA was prepared from one of the transformants. By means of electroporation (Haynes et al., FEMS Microbiology Letters (1989) 61: 329-334), this inactivation plasmid was then introduced into the strain Corynebacterium glutamicum ATCC14752. Clones in which the inactivation vector was present integrated in the genome were identified with the aid of their kanamycin resistance on LBHIS agar plates containing 15 μg/mL kanamycin (Liebl et al., FEMS Microbiology Letters (1989) 65:<<299-304). To select for the excision of the vector, kanamycin-resistant clones were plated out on sucrose-containing LBG medium (LB medium with 15 g/L agar, 2% glucose and 10% sucrose). This gave colonies which have lost the vector again by a second recombination event (Jäger et al.; Journal of Bacteriology (1992) 174: 5462-5465). By transinoculation on to minimal medium plates (CGXII medium with 15 g/L Agar (Keilhauer et al., Journal of Bacteriology (1993) 175: 5595-5603)) with and without 300 mg/L L-isoleucine, or with and without 50 μg/mL kanamycin, six clones were isolated which were kanamycin-sensitive and isoleucine-auxotrophic due to excision of the vector and in which the incomplete ilvA gene (ΔilvA allele) was now present in the genome. One of these clones was designated strain ATCC14752ΔilvA and employed for the transposon mutagenesis.

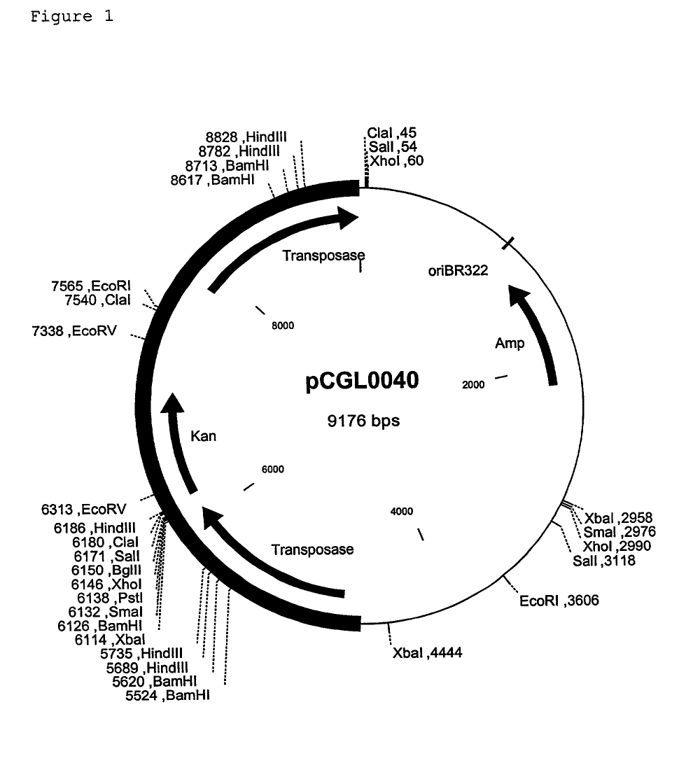

From the methylase-defective E. coli strain GM2929pCGL0040 (E. coli GM2929: Palmer et al., Gene (1994) 143: 1-12) was isolated the plasmid pCGL0040 (FIG. 1), which contains the combined transposon Tn5531 (Ankri et al., Journal of Bacteriology (1996) 178:<<4412-4419). The strain Corynebacterium glutamicum ATCC14752ΔilvA was transformed by means of electroporation (Haynes et al., FEMS Microbiology Letters (1989) 61: 329-334) with the plasmid pCGL0040. Clones in which the transposon Tn5531 was integrated into the genome were identified with the aid of their kanamycin resistance on LBHIS agar plates containing 15 μg/mL kanamycin (Liebl et al., FEMS Microbiology Letters (1989) 65: 299-304). 2000 clones were obtained in this manner, and were investigated for delayed growth in the presence of threonyl-threonyl-threonine. For this, all the clones were transferred individually to CGXII minimal medium agar plates with and without 2 mM threonyl-threonyl-threonine. The medium was identical to the CGXII medium described by Keilhauer et al. (Journal of Bacteriology (1993) 175: 5593-5603), but additionally contained 25 μg/mL kanamycin, 300 mg/L L-isoleucine and 15 g/L agar. The composition of the medium described by Keilhauer et al. is shown in table 1.

TABLE 1
Composition of the CGXII medium

| Component | Concentration |
|---|---|
| (NH₄)₂S04 | 20 g/L |
| Urea | 5 g/L |
| KH₂PO₄ | 1 g/L |
| K₂HPO₄ | 1 g/L |
| MgSO₄ × 7 H₂O | 0.25 g/L |
| 3-Morpholinopropanesulfonic acid | 42 g/L |
| CaCl₂ | 10 mg/L |
| FeSO₄ × 7 H₂O | 10 mg/L |
| MnSO₄ × H₂O | 10 mg/L |
| ZnSO₄ × 7H₂O | 1 mg/L |
| CuSO₄ | 0.2 mg/L |
| NiCl₂ × 6 H₂O | 0.02 mg/L |
| Biotin | 0.2 mg/L |
| Glucose | 40 g/L |
| Protocatechuic acid | 30 mg/L |

The agar plates were incubated at 30° C. and the growth was investigated after 12, 18 and 24 hours. A transposon mutant was obtained which grew in a manner comparable to the starting strain Corynebacterium glutamicum ATCC14752ΔilvA without threonyl-threonyl-threonine but showed delayed growth in the presence of 2 mM threonyl-threonyl-threonine. This was designated ATCC14752ΔilvAthrE::Tn5531.

2. Cloning and Sequencing of the Insertion Site of Tn5531 in ATCC14752ΔilvAthrE::Tn5531

To clone the insertion site lying upstream of the transposon Tn5531 in the mutant described in example 1.1, the chromosomal DNA of this mutant strain was first isolated as described by Schwarzer et al. (Bio/Technology (1990) 9: 84-87) and 400 ng thereof were cleaved with the restriction endonuclease EcoRI. The complete restriction batch was ligated in the vector pUC18 (Norander et al., Gene (1983) 26: 101-106, Roche Diagnostics, (Mannheim, Germany), which was also linearized with EcoRI. The E. coli strain DH5αmcr (Grant et al., Proceedings of the National Academy of Sciences of the United States of America USA (1990) 87: 4645-4649) was transformed with the entire ligation batch by means of electroporation (Dower et al., Nucleic Acid Research (1988) 16: 6127-6145). Transformants in which the insertion sites of the transposon Tn5531 were present cloned on the vector pUC18 were identified with the aid of their carbenicillin and kanamycin resistance on LB agar plates containing 50 μg/mL carbenicillin and 25 μg/mL kanamycin. The plasmids were prepared from three of the transformants and the sizes of the cloned inserts were determined by restriction analysis. The nucleotide sequence of the insertion site on one of the plasmids with an insert approx. 5.7 kb in size was determined by the dideoxy chain termination method of Sanger et al. (Proceedings of the National Academy of Sciences of the United States of America USA (1977) 74: 5463-5467). For this, 2.2 kb of the insert were sequenced starting from the following oligonucleotide primer: 5′-CGG GTC TAC ACC GCT AGC CCA GG-3′.

For identification of the insertion site lying downstream of the transposon, the chromosomal DNA of the mutant was cleaved with the restriction endonuclease XbaI and ligated in the vector pUC18 linearized with XbaI. Further cloning was carried out as described above. The nucleotide sequence of the insertion site on one of the plasmids with an insert approx. 8.5 kb in size was determined by the dideoxy chain termination method of Sanger et al. (Proceedings of the National Academy of Sciences of the United States of America USA (1977) 74: 5463-5467). For this, 0.65 kb of the insert were sequenced starting from the following oligonucleotide primer: 5′-CGG TGC CTT ATC CAT TCA GG-3′.

The nucleotide sequences obtained were analysed and joined together with the Lasergene program package (Biocomputing Software for Windows, DNASTAR, Madison, USA)). This nucleotide sequence is reproduced as SEQ ID NO 1. The result of the analysis was identification of an open reading frame of 1467 bp in length. The corresponding gene was designated the thrE gene. The associated gene product comprises 489 amino acids and is reproduced as SEQ ID NO 2.

### EXAMPLE 2

Cloning and Sequencing of the thrE Gene from Corynebacterium glutamicum ATCC13032

The thrE gene was cloned in the E. coli cloning vector pUC18 (Norrander et al., Gene (1983) 26: 101-106, Roche Diagnostics, Mannheim, Germany). The cloning was carried out in two steps. The gene from Corynebacterium glutamicum ATCC13032 was first amplified by a polymerase chain reaction (PCR) by means of the following oligonucleotide primers derived from SEQ ID NO 1.

thrE-forward:

5′-CCC CTT TGA CCT GGT GTT ATT G-3′

thrE-reverse:

5′-CGG CTG CGG TTT CCT CTT-3′

The PCR reaction was carried out in 30 cycles in the presence of 200 μM deoxynucleotide triphosphates (dATP, dCTP, dGTP, dTTP), in each case 1 μM of the corresponding oligonucleotide, 100 ng chromosomal DNA from Corynebacterium glutamicum ATCC13032, 1/10 volume 10-fold reaction buffer and 2.6 units of a heat-stable Taq-/Pwo-DNA polymerase mixture (Expand High Fidelity PCR System from Roche Diagnostics, Mannheim, Germany) in a Thermocycler (PTC-100, MJ Research, Inc., Watertown, USA) under the following conditions: 94° C. for 30 seconds, 58° C. for 30 seconds and 72° C. for 2 minutes.

The amplified fragment about 1.9 kb in size was then subsequently ligated with the aid of the SureClone Ligation Kit (Amersham Pharmacia Biotech, Uppsala, Sweden) into the SmaI cleavage site of the vector pUC18 in accordance with the manufacturer's instructions. The E. coli strain DH5αmcr (Grant et al., Proceedings of the National Academy of Sciences of the United States of America USA (1990) 87: 4645-4649) was transformed with the entire ligation batch. Transformants were identified with the aid of their carbenicillin resistance on LB agar plates containing 50 μg/mL carbenicillin. The plasmids were prepared from 8 of the transformants and checked for the presence of the 1.9 kb PCR fragment as an insert by restriction analysis. The recombinant plasmid formed in this way is designated pUC18thrE in the following.

The nucleotide sequence of the 1.9 kb PCR fragment in plasmid pUC18thrE was determined by the dideoxy chain termination method of Sanger et al. (Proceedings of the National Academy of Sciences of the United States of America USA (1977) 74: 5463-5467). For this, the complete insert of pUC18thrE was sequenced with the aid of the following primers from Roche Diagnostics (Mannheim, Germany).

Universal Primer:

5′-GTA AAA CGA CGG CCA GT-3′

Reverse Primer:

5′-GGA AAC AGC TAT GAC CAT G-3′

The nucleotide sequence is reproduced as SEQ ID NO 3. The nucleotide sequence obtained was analysed with the Lasergene program package (Biocomputing Software for Windows, DNASTAR, Madison, USA). The result of the analysis was identification of an open reading frame of 1467 bp in length, which was designated the thrE gene.

This codes for a polypeptide of 489 amino acids, which is reproduced as SEQ ID NO 4.

### EXAMPLE 3

Expression of the thrE Gene in Corynebacterium glutamicum

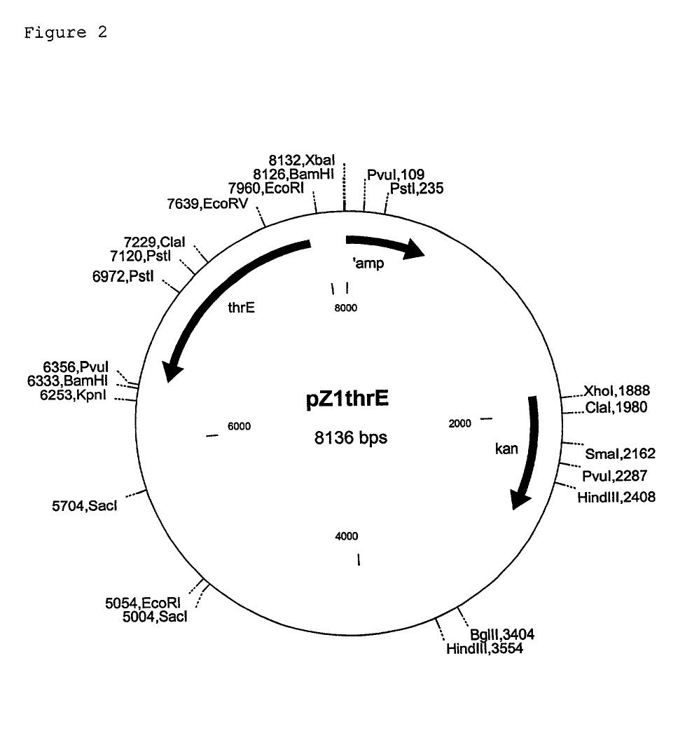

The thrE gene from Corynebacterium glutamicum ATCC13032 described under example 2 was cloned for expression in the vector pZ1 (Menkel et al., Applied and Environmental Microbiology (1989) 64: 549-554). For this, a DNA fragment 1881 bp in size which contained the thrE gene was cut out of the plasmid pUC18thrE with the restriction enzymes SacI and XbaI. The 5′ and 3′ ends of this fragment were treated with Klenow enzyme. The resulting DNA fragment was ligated in the vector pZ1, which was linearized with ScaI and dephosphorylated beforehand. The E. coli strain DH5αmcr (Grant et al., Proceedings of the National Academy of Sciences of the United States of America USA (1990) 87: 4645-4649) was transformed with the entire ligation batch. Transformants were identified with the aid of their kanamycin resistance on LB agar plates containing 50 μg/mL kanamycin. The plasmids were prepared from 2 transformants and checked for the presence of the 1881 bp ScaI/XbaI fragment as an insert by restriction analysis. The recombinant plasmid formed in this manner was designated pZ1thrE (FIG. 2).

By means of electroporation (Haynes et al., FEMS Microbiology Letters (1989) 61: 329-334), the plasmid pZ1thrE was introduced into the threonine-forming strain Brevibacterium flavum DM368-2. The strain DM368-2 is described in EP-B-0 385 940 and deposited as DSM5399. Transformants were identified with the aid of their kanamycin resistance on LBHIS agar places containing 15 μg/mL kanamycin (Liebl et al., FEMS Microbiology Letters (1989) 65: 299-304). The strain Brevibacterium flavum DM368-2 pZ1thrE was formed in this manner.

### EXAMPLE 4

Construction of the Expression Plasmid pTrc99AthrE

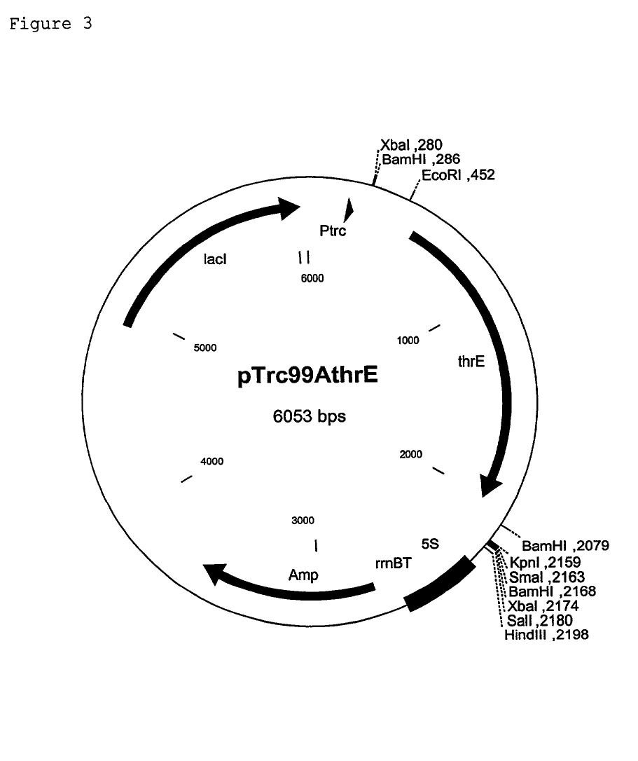

The thrE gene from Corynebacterium glutamicum ATCC13032 described under example 2 was cloned for expression in Escherichia coli in the vector pTrc99A, which was obtained from Pharmacia Biotech (Uppsala, Sweden), for expression in Escherichia coli. For this, the plasmid pUC18thrE was cleaved with the enzyme SalI and the projecting 3′ ends were treated with Klenow enzyme. After restriction with the enzyme KpnI, the cleavage batch was separated in 0.8% agarose gel and the thrE fragment 1.9 kbp in size was isolated with the aid of the QIAquick Gel Extraction Kit (QIAGEN, Hilden, Germany). The vector pTrc99A was cleaved with the enzyme EcoRI, the 3′ ends were treated with Klenow enzyme, cleaved with the enzyme KpnI and ligated with the thrE fragment isolated. The ligation batch was transformed in the E. coli strain DH5α. Selection of cells carrying pTrc99A was carried out on LB agar (Lennox, Virology 1:190 (1955)), to which 50 μg/ml ampicillin had been added. Successful cloning of the thrE gene could be demonstrated after plasmid DNA isolation and control cleavage with XbaI, BamHI. EcoRI, HindIII and SspI. The plasmid was designated pTrc99AthrE (FIG. 3).

### EXAMPLE 5

Preparation of L-threonine with the Strain MG442/pTrc99AthrE

The L-threonine-producing E. coli strain MG442 is described in the patent specification U.S. Pat. No. 4,278,765 and deposited at the Russian National Collection of Industrial Microorganisms (VKPM, Moscow, Russia) as CMIM B-1628.

The strain MG442 was transformed with the plasmid pTrc99AthrE and plasmid-carrying cells were selected on LB agar with 50 μg/ml ampicillin. Selected individual colonies were then multiplied further on minimal medium with the following composition: 3.5 g/l Na₂HPO₄.2H₂O, 1.5 g/l KH₂PO₄, 1 g/l NH₄Cl, 0.1 g/ MgSO₄.7H₂O, 2 g/l glucose, 20 g/l agar, 50 mg/l ampicillin. The formation of L-threonine was checked in batch cultures of 10 ml contained in 100 ml conical flasks. For this, 10 ml preculture medium of the following composition: 2 g/l yeast extract, 10 g/l (NH₄)2SO₄, 1 g/l KH₂PO₄, 0.5 g/l MgSO₄.7H₂O, 15 g/l CaCO₃, 20 g/l glucose, 50 mg/l ampicillin were inoculated and incubated for 16 hours at 37° C. and 180 rpm on an ESR incubator from Kühner AG (Birsfelden, Switzerland). 250 μl portions of this preculture were transinoculated into 10 ml of production medium (25 g/l (NH₄)2SO₄, 2 g/l KH₂PO₄, 1 g/1 MgSO₄.7H₂O, 0.03 g/l FeSO₄.7H₂O, 0.018 g/l MnSO₄.1H₂O, 30 g/l CaCO₃, 20 g/l glucose) and the mixtures were incubated for 48 hours at 37° C. For induction of the expression of the thrE gene, 200 mg/l isopropyl β-D-thiogalactoside (IPTG) were added in parallel batches. After the incubation, the optical density (OD) of the culture suspension was determined with an LP2W photometer from Dr. Lange (Berlin, Germany) at a measurement wavelength of 660 nm.

The concentration of L-threonine formed was then determined in the sterile-filtered culture supernatant with an amino acid analyser from Eppendorf-BioTronik (Hamburg, Germany) by ion exchange chromatography and post-column reaction with ninhydrin detection.

The result of the experiment is shown in table 2.

TABLE 2

| | | | L-Threonine |
|---|---|---|---|
| Strain | Additives | OD | g/l |
| MG442 | — | 5.6 | 1.38 |
| MG442/pTrc99AthrE | — | 4.6 | 1.65 |
| MG442/pTrc99AthrE | IPTG | 3.6 | 3.5 |

### EXAMPLE 6

Preparation of L-threonine with the Strain B-3996kurΔtdh/pVIC40, pMW218thrE

The L-threonine-producing E. coli strain B-3996 is described in U.S. Pat. No. 5,175,107 and deposited at the Russian National Collection for Industrial Microorganisms (VKPM, Moscow, Russia).

6.1 Cloning of the thrE Gene in the Plasmid Vector pMW218

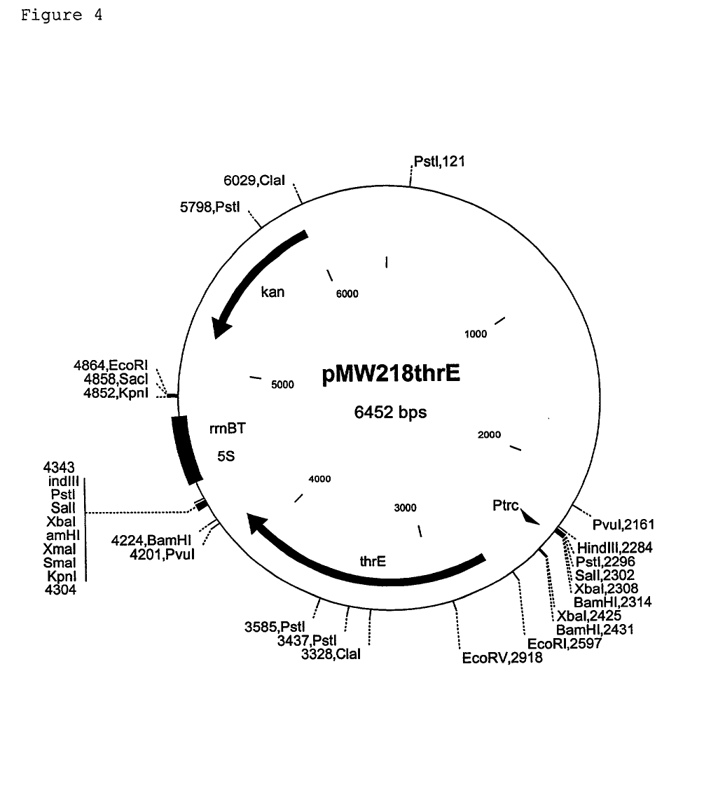

The plasmid pTrc99AthrE described under example 4 was cleaved with the enzyme SspI, the cleavage batch was separated in 0.8% agarose gel and the DNA fragment 2.5 kbp in size, which contained the trc promoter region and the rRNA terminator region in addition to the thrE gene, was isolated with the aid of the “QIAquick Gel Extraction Kit” (QIAGEN, Hilden, Germany). The plasmid pMW218 (Nippon Gene, Toyama, Japan) was cleaved with the enzyme SmaI and ligated with the thrE fragment. The E. coli strain DH5α was transformed with the ligation batch and pMW218-carrying cells were selected by plating out on LB agar, to which 20 μg/ml kanamycin are added. Successful cloning of the thrE gene could be demonstrated after plasmid DNA isolation and control cleavage with HindIII and ClaI. The plasmid was designated pMW218thrE (FIG. 4).

6.2 Preparation of the Strain B-3996kurΔtdh/pVIC40, pMW218thrE

After culture in antibiotic-free complete medium for approximately ten generations, a derivative of strain B-3996 which no longer contained the plasmid pVIC40 was isolated. The strain formed was streptomycin-sensitive and was designated B-3996kur.

The method described by Hamilton et al. (Journal of Bacteriology (1989) 171: 4617-4622), which is based on the use of the plasmid pMAK705 with a temperature-sensitive replicon, was used for incorporation of a deletion into the tdh gene. The plasmid pDR121 (Ravnikar and Somerville, Journal of Bacteriology (1987) 169: 4716-4721) contains a DNA fragment from E. coli 3.7 kilo-base pairs (kbp) in size, on which the tdh gene is coded. To generate a deletion of the tdh gene region, pDR121 was cleaved with the restriction enzymes ClaI and EcoRV and the DNA fragment 5 kbp in size isolated was ligated, after treatment with Klenow enzyme. The ligation batch was transformed in the E. coli strain DH5α and plasmid-carrying cells were selected on LB agar, to which 50 μg/ml ampicillin are added.

Successful deletion of the tdh gene could be demonstrated after plasmid DNA isolation and control cleavage with EcoRI. The EcoRI fragment 1.7 kbp in size was isolated, nd ligated with the plasmid pMAK705, which was partly digested with EcoRI. The ligation batch was transformed in DH5α and plasmid-carrying cells were selected on LB agar, to which 20 μg/ml chloramphenicol were added. Successful cloning was demonstrated after isolation of the plasmid DNA and cleavage with EcoRI. The pMAK705 derivative formed was designated pDM32.

For the gene replacement, B-3996kur was transformed with the plasmid pDM32. The replacement of the chromosomal tdh gene with the plasmid-coded deletion construct was carried out by the selection process described by Hamilton et al. and was verified by standard PCR methods (Innis et al. (1990), PCR Protocols. A Guide to Methods and Applications, Academic Press) with the following oligonucleotide primers:

| | |
|---|---|
| Tdh1: | 5′-TCGCGACCTATAAGTTTGGG-3′ |
| Tdh2: | 5′-AATACCAGCCCTTGTTCGTG-3′ |

The strain formed was tested for kanamycin sensitivity and was designated B-3996kurΔtdh.

B-3996kurΔtdh was transformed with the plasmid pVIC40 isolated from B-3996 and plasmid-carrying cells were selected on LB agar supplemented with 20 μg/ml streptomycin. A selected individual colony was designated B-3996kurΔtdh/pVIC40 and transformed with the plasmid pMW218thrE. Selection is carried out on LB-agar to which 20 μg/ml streptomycin and 50 μg/ml kanamycin are added. The strain formed in this way was designated B-3996kurΔtdh/pVIC40, pMW218thrE.

6.3 Preparation of L-threonine

The preparation of L-threonine by the strains B-3996kurΔtdh/pVIC40 and B-3996kurΔtdh/pVIC40, pMW218thrE was tested as described in example 5. The minimal medium, the preculture medium and the production medium were supplemented with 20 μg/ml streptomycin for B-3996kurΔtdh/pVIC40 and with 20 μg/ml streptomycin and 50 μg/ml kanamycin for B-3996kurΔtdh/pVIC40, pMW218thrE.

The result of the experiment is summarized in table 3.

TABLE 3

| | OD | L-Threonine |
|---|---|---|
| Strain | (660 nm) | g/l |
| B-3996kurΔtdh/pVIC40 | 4.7 | 6.26 |
| B-3996kurΔtdh/pVIC40, | 4.8 | 7.57 |
| pMW218thrE | | |

The following figures are attached:

FIG. 1: Map of the plasmid pCGL0040 containing the transposon Tn5531. The transposon is identified as the non-shaded arrow.

FIG. 2: Map of the plasmid pZ1thrE containing the thrE gene.

FIG. 3: Map of the plasmid pTrc99AthrE containing the thrE gene.

FIG. 4: Map of the plasmid pMW218thrE containing the thrE gene

The length data are to be understood as approx. data. The abbreviations and designations used have the following meaning:

Amp: Ampicillin resistance gene

Kan: Kanamycin resistance gene

'amp: 3′ part of the ampicillin resistance gene

oriBR322: Replication region of the plasmid pBR322

lacI: Gene for the repressor protein of the trc promoter

Ptrc: trc promoter region, IPTG-inducible

5S: 5S rRNA region

rrnBT: rRNA terminator region

The abbreviations for the restriction enzymes have the following meaning

BamHI: Restriction endonuclease from Bacillus amyloliquefaciens

BglII: Restriction endonuclease from Bacillus globigii

ClaI: Restriction endonuclease from Caryphanon latum

EcoRI: Restriction endonuclease from Escherichia coli

EcoRV: Restriction endonuclease from Escherichia coli

HindIII: Restriction endonuclease from Haemophilus influenzae

KpnI: Restriction endonuclease from Klebsiella pneumoniae

PstI: Restriction endonuclease from Providencia stuartii

PvuI: Restriction endonuclease from Proteus vulgaris

SacI: Restriction endonuclease from Streptomyces achromogenes

SalI: Restriction endonuclease from Streptomyces albus

SmaI: Restriction endonuclease from Serratia marcescens

XbaI: Restriction endonuclease from Xanthomonas badrii

XhoI: Restriction endonuclease from Xanthomonas holcicola

1. A process for the fermentative preparation of L-threonine, which comprises employing Enterobacteriaceae bacteria, in particular those which already produce L-threonine and in which the nucleotide sequence(s) of coryneform bacteria which code(s) for the thrE gene are enhanced, in particular over-expressed.

2. A process as claimed in claim 1, wherein further genes are enhanced in addition to the thrE gene.

3. A process as claimed in claim 1 or 2, wherein the microorganisms of the family Enterobacteriaceae are from the genus Escherichia and Serratia.

4. A process as claimed in claim 3, wherein the microorganisms are from the genus Escherichia, in particular of the species Escherichia coli.

5. A process as claimed in claim 1, wherein the thrABC operon which codes for aspartate kinase, homoserine dehydrogenase, homoserine kinase and threonine synthase is enhanced at the same time.

6. A process as claimed in claim 1, wherein the pyc gene which codes for pyruvate carboxylase is enhanced at the same time.

7. A process as claimed in claim 1, wherein the pps gene which codes for phosphoenol pyruvate synthase is enhanced at the same time.

8. A process as claimed in claim 1, wherein the ppc gene which codes for phosphoenol pyruvate carboxylase is enhanced at the same time.

9. A process as claimed in claim 1, wherein the genes pntA and pntB which code for transhydrogenase are enhanced at the same time.

10. A process as claimed in claim 1, wherein bacteria in which the metabolic pathways which reduce the formation of L-threonine are at least partly eliminated are employed.

11. A process as claimed in claim 1, wherein a strain transformed with a plasmid vector is employed and the plasmid vector carries the nucleotide sequence which codes for the thrE gene of coryneform bacteria.

12. A process as claimed in claim 1, wherein bacteria transformed with the plasmid pZ1thrE are employed.

13. A process as claimed in claim 1, wherein the expression of the thrE gene is induced, in particular with isopropyl β-D-thiogalactoside.

14. A process as claimed in claim 1, wherein the gdhA gene which codes for glutamate dehydrogenase is enhanced at the same time.

15. A process as claimed in claim 1, wherein the rhtB gene which imparts homoserine resistance is enhanced at the same time.

16. A process for the preparation of L-threonine, which comprises carrying out the following steps:
a) fermentation of microorganisms of the family Enterobacteriaceae in which at least the thrE gene of coryneform bacteria is enhanced (over-expressed), optionally in combination with further genes,
b) concentration of the L-threonine in the medium or in the cells of the microorganisms of the family Enterobacteriaceae, and
c) isolation of the L-threonine.

17. The plasmid pZ1thrE which contains the thrE gene of Corynebacterium glutamicum ATCC13032.

18. The Brevibacterium flavum strain DM368-2 pZ1thrE deposited as DSM 12840 at the DSMZ [German Collection of Microorganisms and Cell Cultures], Braunschweig.
